(19) United States
(12) Patent Application Publication
(10) Pub. No.: US 2025/0369853 A1
(43) Pub. Date: Dec. 4, 2025

(54) Methods and Apparatus for Review of Proppant Transport

(71) Applicant: CANADIAN ENERGY SERVICES L.P., Calgary (CA)

(72) Inventors: Coleman STEPHENS, Calgary (CA); David HORTON, Calgary (CA)

(21) Appl. No.: 19/218,882

(22) Filed: May 27, 2025

Related U.S. Application Data
(60) Provisional application No. 63/653,083, filed on May 29, 2024.

(51) Int. Cl. G01N 15/075; G01N 15/00; G01N 33/28

(52) U.S. Cl. CPC G01N 15/075; G01N 33/2823; G01N 2015/0053

(57) ABSTRACT

An apparatus and method to test particulate suspension in a liquid. The apparatus includes: a hollow cylindrical container including a transparent wall defining therein an open cylindrical area; and a member including a solid cylindrical outer wall, the member positionable within the open cylindrical area with a gap between the solid cylindrical outer wall and the transparent wall, the hollow cylindrical container and the member being configured so that liquid to be tested is contained in the open cylindrical area between the member and the hollow cylindrical container and either the hollow cylindrical container or the member is configured to be rotated.

### FIELD

The invention relates to methods and apparatus for review of particulate solid suspension in liquids, such as in wellbore treatment fluids.

### BACKGROUND

Wellbore treatment fluids must be able to carry the proppant contained therein down into the wellbore and into a fracture network extending from the wellbore. The efficiency of proppant suspension and transport of the wellbore treatment fluids must therefore be known.

To be clear, proppant is a particulate solid that is used in wellbore treatments, such as hydraulic fracturing treatments, to facilitate the creation of, and to hold open, fractures in the geological formation through which the wellbore extends. Proppant is usually rock particles such as sand, but may be resin, ceramic, shell and other hard solids. Fracturing fluid is a liquid chemical mixture that is used in hydraulic fracturing treatments.

For hydraulic fracturing treatments, fracture efficiency is dependent on the proficiency of proppant transport into the fracture aperture. Resultant fracture conductivity is dependent on how well proppant can be distributed throughout the fracture prior to depletion of the fracturing injection pressure. Typically, two mechanisms are present that can assist with proppant transport: inertial effects and rheological effects. Within the wellbore, at high Reynolds numbers, inertial effects are the dominant mechanism for proppant transport. Within the reservoir, the rheological properties of the fluid govern proppant diffusion, along with fracture geometry and fluid leak off.

Dynamic Proppant Suspension (DPS) testing has historically employed a mix of experimental, theoretical, and numerical approaches, with a focus on understanding fluid-proppant interactions under various conditions. DPS has been characterized utilizing several different methods such as settling tests based upon Stokes Law, slot-flow tests and fracture simulators, traditional viscometers and others. Other methods such as settling tests based upon Stokes Law have also been employed with particle image velocimetry measurements to ensure precise tracking and analysis of proppant settling behaviors with Newtonian fluids in the absence of shear.

### SUMMARY

There is provided a method and apparatus for determining a suspension characteristic of a particulate solid in a liquid, such as but not limited to a proppant in a wellbore fluid.

According to one aspect, there is provided an apparatus to test particulate suspension in a liquid, the apparatus comprising: a hollow cylindrical container including a transparent wall defining therein an open cylindrical area; and a member including a solid cylindrical outer wall, the member positionable within the open cylindrical area with a gap between the solid cylindrical outer wall and the transparent wall, the hollow cylindrical container and the member being configured so that liquid to be tested is contained in the open cylindrical area between the member and the hollow cylindrical container and either the hollow cylindrical container or the member is configured to be rotated.

According to another broad aspect, there is provided a method for testing particulate suspension in a liquid using the apparatus above, the method comprising: introducing the liquid and a particulate to the gap; rotating either the hollow cylindrical container or the member such that a shear field is induced between the member and the hollow cylindrical container; and monitoring the particulate suspension of the particulate in the liquid.

### BRIEF DESCRIPTION OF DRAWINGS

FIG. 1 is a schematic side elevation of an apparatus according to the invention installed with a motor ready for use.

FIG. 2 is a section along line I-I, which is orthogonal to the center axis of the apparatus, of FIG. 1.

FIGS. 3 to 7 are results and analysis from Example I.

### DETAILED DESCRIPTION OF THE INVENTION

The present invention relates to an apparatus and a method for studying a suspension characteristic of a particulate solid in a liquid. In one embodiment, the apparatus and method are for quantifying DPS in hydraulic fracturing.

Understanding the mechanisms of DPS has been heavily debated within the oil and gas industry where many authors and papers have set up empirical experiments, theoretical or numerical studies to understand the prevalent mechanisms for designing a fluid and polymer-based system to maximize proppant transport. Two mechanisms that are heavily contested are inertial effects and rheological effects. A pre-dominant view in the industry is that one of the most important parameters for dynamic suspension is viscosity, measured at the shear rate in the fracture during pumping between 10 to 100 s⁻¹. The explanation for this parameter has originated from Stokes Law, which states that sedimentation is inversely proportional to medium viscosity. One limitation of Stoke's Law is that it applies to low Reynolds number conditions where the flow is laminar and a Newtonian fluid. Stoke's law also assumes that particles do not interfere with each other, and studies are typically performed on single particle designs, which is not representative of fracturing fluid systems. If the flow becomes turbulent at Reynolds numbers greater than 4000, Stokes law no longer provides an accurate description of the settling velocity. Recent laboratory work has shown that viscosity alone may not accurately assess proppant transport, with more studies and experiments analyzing rotational and oscillatory measurements to determine the viscous and elastic properties of the fluid.

Most experimental studies pertain to conditions where viscosity is a function of the shear rate whereas most theoretical developments model the effect of fluid viscoelasticity on spheres in the absence of shear thinning effects. Incorporating a realistic description of shear rate dependent viscosity together with fluid viscoelasticity has been a challenge in theoretical developments.

To prevent settling, the common practice is to use high viscosity friction reduction additives (HVFR's) to keep the proppant material suspended to allow it to penetrate further into the fractures. Prior to high viscosity fracturing fluids, in slick water systems, due to the low viscosity of the fluid, the placement of higher density proppants and higher proppant concentrations was difficult. As such, very high pumping rates are employed to transport the proppant into the fracture by velocity rather than the fluid viscosity and elasticity.

Suspension or proppant carrying capacity is poor in slick water, which has evolved from conventional polyacrylamides as friction reduction additives to current HVFR's. These limitations within DPS showcase that low-polymer concentration fracturing fluids should be characterized with methods that use realistic shear-rate and shear-rate history. These physical factors influence viscouselastic fluid properties as much as chemistry, and determine the fluid's ability to generate fracture geometry and transport proppant. Transport can be defined as the ability of a fluid to carry sand from one point to another without settling. The findings show that the generally accepted test criteria used for selection of HVFR's for field use, does not provide sufficient indication of the product's ability to suspend and transport sand.

Too much viscosity has also been found to have an impact on the ability of fluids to properly suspend and transport sand leading to formation damage from dosage or type of polymer used. Previous research suggest that viscosity is not the primary factor influencing proppant transport. In most cases, products with comparable viscosity had very different performance results. The results provided insight into the effect of flow rate on proppant transport, with some HVFR's that exhibited higher viscosities at low shear, losing their transport capacity at the same low shear. Elasticity testing of those same products suggested that HVFR's have a critical elasticity range at which they will provide optimal DPS. Elasticity testing suggests a potential for better identification of key performance criteria and requires further evaluations. Viscosity should not be used as the sole parameter for determination of a product's ability to suspend and transport sand. Furthermore, it should not be the criteria used to compare one product versus another. Evaluation of the suspension properties of bulk particles under dynamic conditions along with elastic properties of the fluid can provide a better understanding of the performance ranges for conventional friction reduction (FR) and HVFR products.

The present invention provides methods and apparatus using a modified rotational coaxial cylinder viscometer. The invention also optionally provides an image processing technology to precisely measure particulate solid settling rates under specific shear conditions. In such an embodiment, high-speed cameras can be employed to assess particulate solid transport dynamics, for example with a focus on the interplay between fluid dynamics, particulate characteristics, and chemical properties.

In one embodiment, the invention aims to provide methodologies and tools to optimize proppant transport efficiency in fracturing fluids, to permit high viscosity friction reducing additives (HVFR's) to be studied, selected or engineered with a better understanding of their performance. By highlighting the significance of inertial and rheological effects across various shear rates and the impact of fracture geometry on proppant transport, the invention can be employed to enhance fracture efficiency and conductivity. The current method considers proppant behavior under varying conditions.

The methodology allows for the inclusion of high concentrations of proppant, which is better than single particle, static settling test often modeled utilizing Stoke's law for settling velocity.

Referring to the Figures, an apparatus 10 to test and monitor liquids according to the invention includes a member 12 and a hollow cylindrical container 20. The apparatus is configured so that liquid to be tested can be introduced to the hollow cylindrical container and the member 12 can be inserted into the hollow cylindrical container and placed into the liquid within the container and one or the other of the container and the member can be rotated while the other of the container or the member remains static. Then, the reaction of the liquid to the shear effect between the rotating part and the static, non-rotating part can be assessed.

In the following description, the member 12 is sometimes described as being rotated, while the container 20 remains static. It is to be understood, however, that the opposite, where member 12 is static and container 20 is rotated about the member, can be employed as well.

In one embodiment, member 12 has a solid cylindrical outer wall 12a and a shank 12b. The solid cylindrical outer wall is devoid of protrusions, cavities, holes, paddles, etc. such that the full circumference of the solid cylindrical outer wall is a smooth, solid, cylindrically curved surface.

Hollow cylindrical container 20 has an inner open cylindrical area defined within a closed bottom and a cylindrical wall 20a with an inner facing surface 20a′. The inner facing surface of solid cylindrical wall 20a is devoid of protrusions, cavities, holes, etc. such that the full inner facing cylindrical wall is a smooth, solid cylindrically curved surface. In one embodiment, at least the cylindrical wall 20a of the hollow cylindrical container 20 is constructed of a transparent material such that it is possible to see through the wall into the open cylindrical area. If the wall 20a is transparent, computer imaging can be utilized to observe the condition of the fluid within the container, as will be appreciated by reference to the method below. The inner open cylindrical area has a radius r₂₀.

Radius r₂₀ is greater than a radius r₁₂ of the cylindrical outer wall 12a of member 12, such that the member's solid cylindrical outer wall 12a can fit into the inner open cylindrical area 20a with an annular gap g between the parts. The gap g is narrow. For example, gap g may be about three to ten times the maximum particle size of the particulate solid, for example proppant, to be tested. Proppant is typically between 106 μm and 2.5 mm. In one embodiment, the r₁₂/r₂₀ is a maximum of 0.90, for example, 0.97 or less.

As noted, in operation, there is relative rotation between the container and the member: either the container or the member is rotated while the other of the container or the member remains static. Rotational speeds of up to 600 rpm are of interest.

The apparatus can be employed with a driver to drive this relative rotation or, optionally, the apparatus can include a motor 24 to drive the relative rotation. While the motor 24 is illustrated as configured to engage the shank 12b to rotate R the solid cylindrical outer wall 12a or possibly the whole rotatable member 12, motor 24 can alternatively be configured to rotate container 20. The motor can have a variable speed drive with feedback capabilities. In one embodiment, the motor can have a controller 26 that drives rotation at any speed between 0 and 600 rpm. The controller 26 can also be configured to change the rate of rotation over time.

The apparatus can be based on a Couette viscometer. A commercially available Couette viscometer is the Model 35 Viscometer from Fann Instrument Company, Houston, Texas (fann.com). Viscometers are also available from OFI Testing Equipment Inc.

Starting with a Couette viscometer, a hollow, transparent cylindrical container 20 is selected and a member 12 with a solid cylindrical outer wall 12a is selected that can fit within the container.

This device may be employed in a method of determining dynamic solids suspension characteristics of liquids. A method includes:
1) Configuring a rotatable member 12 to be rotated about a long axis of a cylindrical outer wall 12a at a speed of up to 600 rpm. In particular, the rotatable member 12 may be coupled for receiving drive input from a motor that is configured to rotate the member at any speed greater than 0 rpm and up to 600 rpm.
2) Placing cylindrical outer wall 12a of rotatable member 12 concentrically into a hollow cylindrical container 20 with a radius that is greater than that of solid cylindrical outer wall 12a.
3) Before or after step 2, adding fluid to be tested to the hollow cylindrical container 20. The volume of fluid is such that the solid cylindrical wall is immersed at least in part in the fluid. The solid cylindrical outer wall 12a of the rotatable member 12 is then rotated while the hollow cylindrical container 20 is held stationary. The rotation is at a rate such that a shear field is induced between rotatable member 12 and the hollow cylindrical container 20 sufficient to keep the solid particulate from settling. The fluid to be tested contains a base liquid and a solid particulate, for example, proppant. The solid particulate may be added to the base liquid prior to adding the fluid to the hollow cylindrical container. In such an embodiment, the base liquid and particulate may be pre-mixed. Otherwise, the solid particulate may be added to the base liquid after it is introduced to the container. In one embodiment, the solid particulate may be added to the base liquid after rotating has been initiated.
5) The suspension properties of the solid particulate in the base liquid are monitored during rotation. For example, the solid particulate is monitored to determine if and to what degree it remains suspended in the base liquid. Monitoring includes human observation and/or computer imaging. The input rotational speed of member 12 can be adjusted to emulate reservoir shear conditions. Settling rates may be acquired, for example at regular intervals or continuously, and the data can be analyzed temporally to study the shear effects on the fluid over time. For example, in one embodiment, the rotational velocity of the rotating solid cylindrical outer wall 12a is changed while the system is monitored. For example, the rotational velocity of the rotating solid cylindrical outer wall 12a can be sped up or slowed down. In one embodiment, this may include programming a logic controller of the motor to slowly ramp down or ramp up the rotational velocity of solid cylindrical outer wall 12a. In this embodiment, it is desirable to analyze dynamic suspension efficacy over a range of shear rates, i.e. over a range of rotational speeds. Monitoring is facilitated where there is a colour contrast between the base liquid and the solid particulate. Generally, systems where the base liquid is lighter coloured than the particulate.
6) Then the method may include (i) determining the rotational velocity, and hence the shear rate, at which insufficient shear is present to suspend the particulate and/or (ii) determining the rotational velocity, and hence the shear rate, at which sufficient shear is present to suspend settled particulate. The cross-sectional area of the fluid column that contains a certain threshold of particulate may be calculated.

While the above method describes the member 12 as rotating versus the container 20, it will be appreciated that the same method can be employed but with the container 20 rotating about the member 12 that is inserted therein. In some embodiments, it is preferred to employ a rotating outer container 20, while the inner member 12 remains stationary. This is the configuration of a Couette geometry viscometer.

Various methods can be employed to monitor the suspension of the particulate in the base liquid. By use of a transparent container 20, the operator may view the state of the base liquid and the suspension of particulate therein. In other words, the operator may watch for settling of the particulate in the base liquid.

In one test procedure, a critical settling point may be determined. Critical settling point is defined as a coordinate of time and shear whereby further change in the shear rate has minimal effect on the degree to which the particulate is suspended in the base liquid. The critical settling point could apply in a test procedure where the shear rate is being gradually increased or decreased. For example, based on initial testing, it may be the case that a critical settling point will be met as rotational velocity is decreased, and there may be a point at which further reductions in rotational velocity do not result in additional reductions in particulate suspension.

Using the method and apparatus, the shear rate can be determined readily. The shear rate can be calculated using the tangential velocity and the gap between the cylinders. Tangential velocity is the linear speed of any point on a rotating object, moving along a circular path, where v=r* [symbol] (v=tangential velocity, r=radius of the circle, [symbol] =rotational velocity). Shear rate for the fluid in the annular gap g is calculated by dividing the tangential velocity of the rotating part by the gap g between the solid cylindrical outer wall 12a and the hollow cylindrical container 20, which is the difference between r₂₀ and r₁₂.

The degree to which particulate is suspended in the base liquid can be observable by a person. Gradations may be added to (i.e. marked on, applied to, etc.) the transparent outer cylinder to quantify the degree of particulate suspension. The gradations are marked at a series of spaced intervals from the closed bottom.

Computer imaging is alternatively useful to monitor the suspension of the particulate in the base liquid. In one embodiment, computer imaging is employed to monitor the proppant suspension. For example, an imaging device 28 and imaging software such as an open source image processing package called Fiji, available from the ImageJ website (https://imagej.net/software/fiji/) may be used for imaging with the apparatus. For example, based on image data obtained by device 28, the software is configured to calculate the cross-sectional area of the fluid within the concentric cylinder volume or gap that is considered to contain a critical amount of substrate/proppant, or the “propped area”. The imaging processing utility dissects a video of the apparatus in operation into a number of frames. Each frame is subsequently dissected into pixels. Based on a colour threshold input by the user, each pixel of each frame can be categorized binarily into black or white. In this case, with the proppant substate being dark and the fluid and apparatus equipment within the frame range being light colored, if a pixel is considered to be darker than the input threshold, then it will be considered to be a black pixel. The black pixels are aggregated into the “propped area” as a ratio of total pixels in the frame. The software can discretize the input videos into frames; each frame is then further discretized into elements; the image is converted to an 8-bit format; an input threshold is provided and each element on every frame is then converted to binary based on the threshold; finally, the binary matrix is converted to the “propped area”.

### Example I

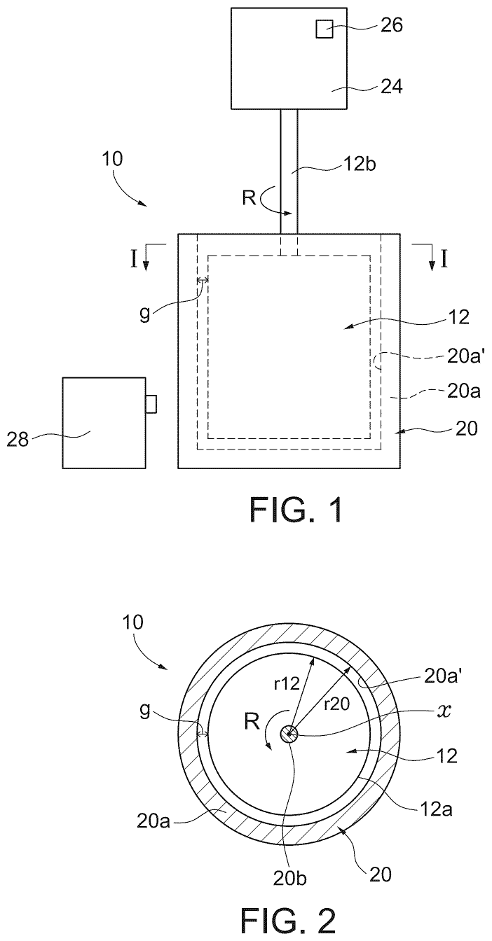

The cross-sectional area of the proppant laden fracturing fluid slurry, guar and various other fluids were measured using the present method. The test utilized a Brookfield PVS rheometer that was equipped with an apparatus as in FIGS. 1 and 2 with a transparent container 20.

Two proppant suspensions were analyzed: one containing HVFR PFR-Z™ (available from PureChem Services Ltd., a division of Canadian Energy Services, Canada) and the second being FR-2 fracturing fluid. Proppant Loading: 500 kg/m³ and HVFR Loading: 4 L/m³ in water.

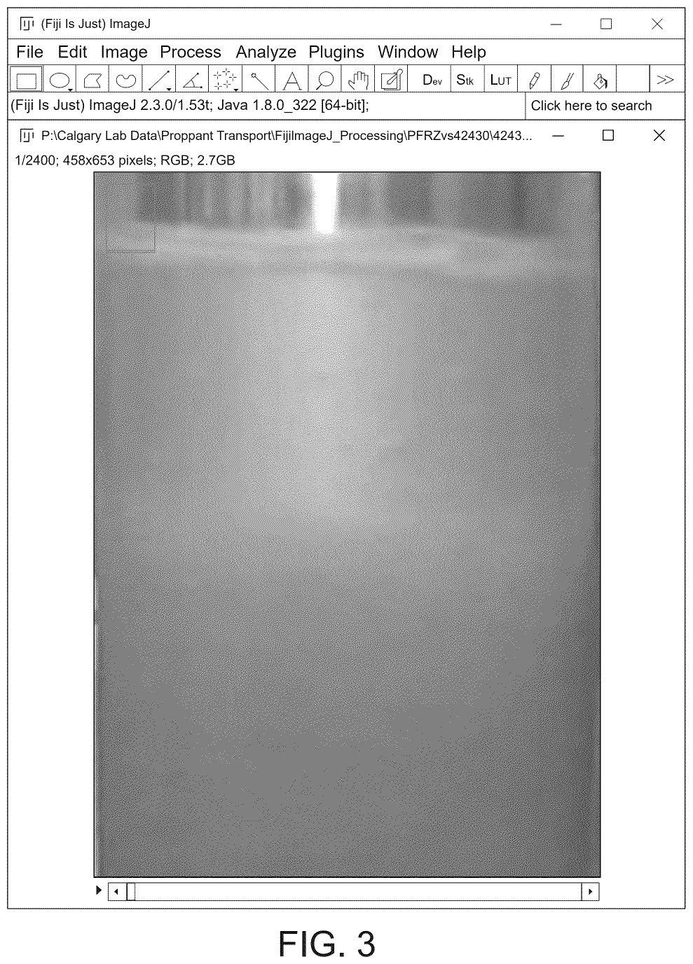

As shown in FIG. 3, videos of the cross-section of the proppant laden slurry were captured using a high-definition camera while measuring radial deflection and then converting to viscosity. Utilizing the Brookfield PVS rheometer and the apparatus as in FIGS. 1 and 2, the shear rate/RPM and chemical loading on dynamic proppant suspension can be accurately measured. The transparent container allowed visual processing.

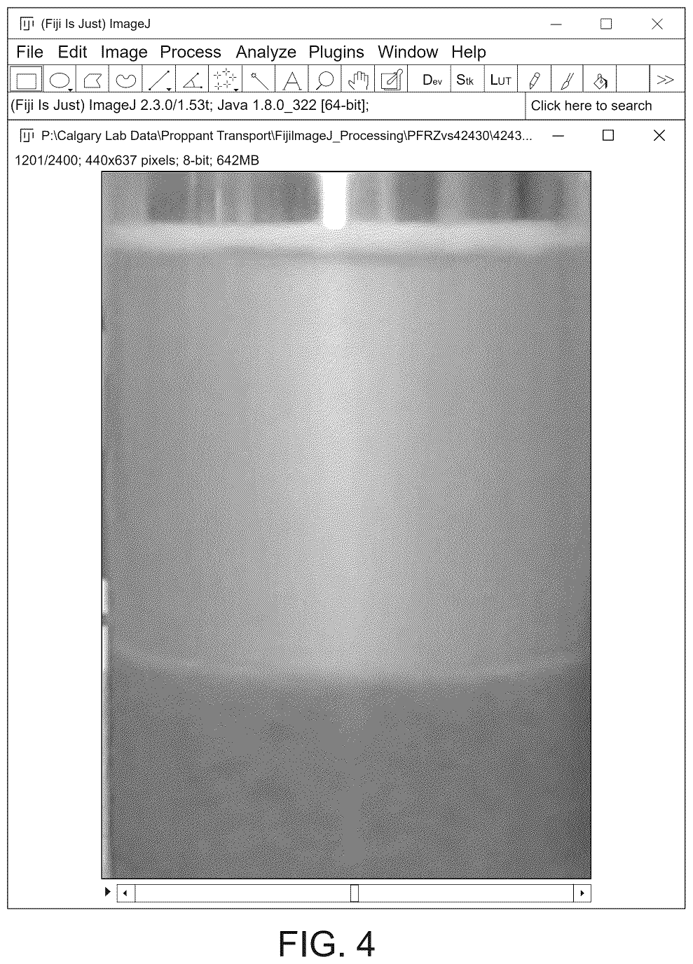

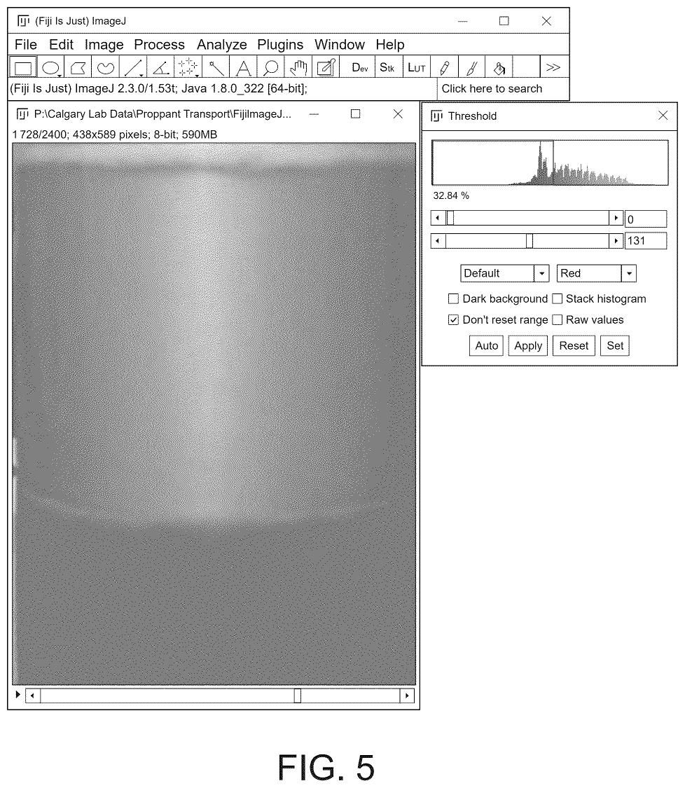

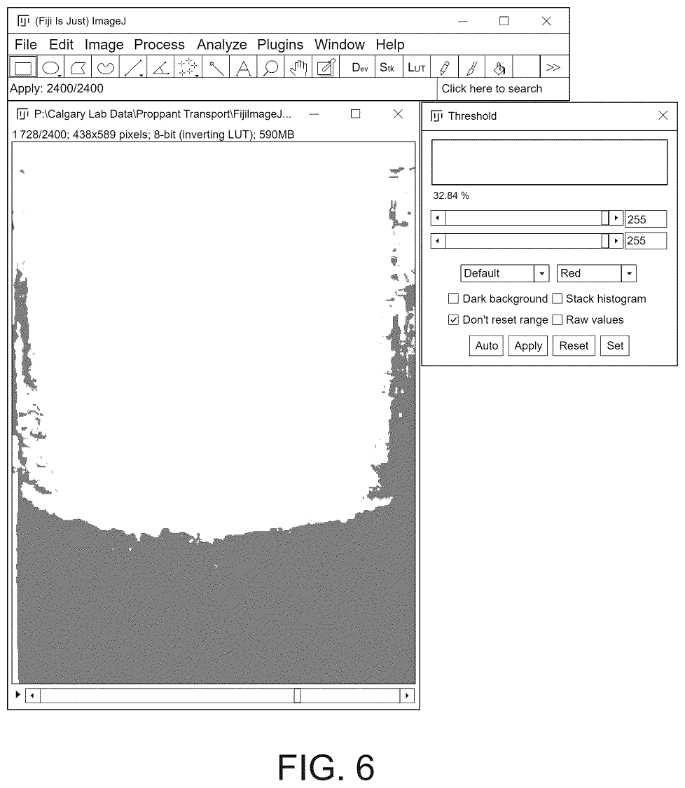

Open-source image processing package called Fiji-based on ImageJ2-was used to quantify the degree of proppant settling. In this case, the videos were converted to 8-bit greyscale (FIG. 4) and then segmented into features of interest and background using global thresholding (FIG. 5). Using a standardized threshold cutoff, the image pixels were converted to binary data, as containing proppant or not (FIG. 6), where gray pixels contain a threshold of proppant.

The processing was completed in bulk on every frame of the video. A frame rate of 8 was used (8 frames per second).

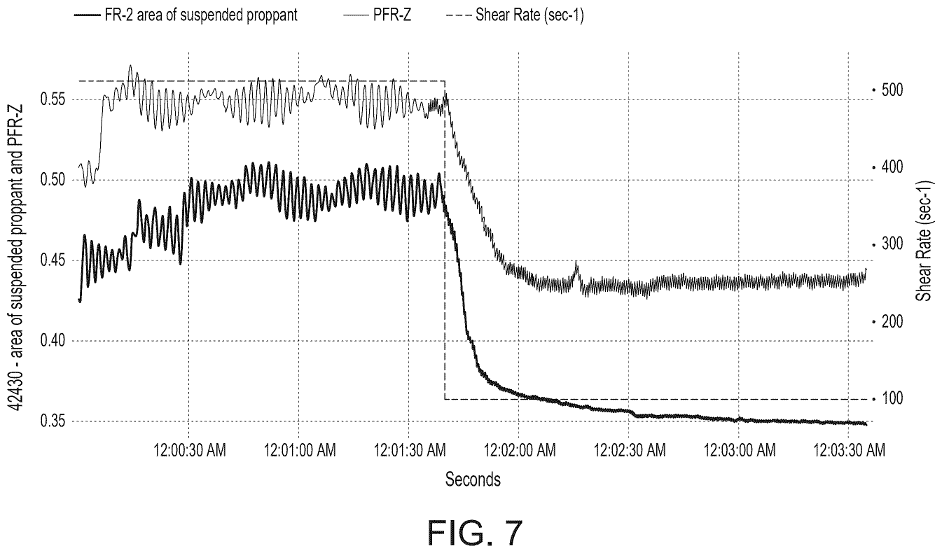

The output from the analysis is a table that provides the number of pixels that are above threshold (contain proppant) for every frame. The pixel amount is converted to a ratio of the overall area and plotted against shear rate (FIG. 7).

The PFR-Z fluid suspended proppant more effectively across the entire shear regime: 511 sec⁻¹ and 99 sec⁻¹. At the lower shear of 99 sec⁻¹, the PFR-Z maintained a suspension rate of ˜44% of the cross-sectional area of the cup, while the FR-2 saw continued drop-out.

The methodology provides a replicable analysis tool for quantifying the rate of proppant transport at known shear rates. The shear rate was known and controllable. With a known shear rate, the data can be used to interpolate proppant suspension within a fracture. Furthermore, proppant dropout can be accurately determined to a specific shear rate.

The previous description of the disclosed embodiments is provided to enable any person skilled in the art to make or use the present invention. Various modifications to those embodiments will be readily apparent to those skilled in the art, and the generic principles defined herein may be applied to other embodiments without departing from the spirit or scope of the invention. Thus, the present invention is not intended to be limited to the embodiments shown herein, but is to be accorded the full scope consistent with the claims, wherein reference to an element in the singular, such as by use of the article “a” or “an” is not intended to mean “one and only one” unless specifically so stated, but rather “one or more”. All structural and functional equivalents to the elements of the various embodiments described throughout the disclosure that are known or later come to be known to those of ordinary skill in the art are intended to be encompassed by the elements of the claims. Moreover, nothing disclosed herein is intended to be dedicated to the public regardless of whether such disclosure is explicitly recited in the claims. No claim element is to be construed under the provisions of 35 USC 112, sixth paragraph, unless the element is expressly recited using the phrase “means for” or “step for”.

1. An apparatus to test particulate suspension in a liquid, the apparatus comprising:
a hollow cylindrical container including a transparent wall defining therein an open cylindrical area; and
a member including a solid cylindrical outer wall, the member positionable within the open cylindrical area with a gap between the solid cylindrical outer wall and the transparent wall,
the hollow cylindrical container and the member being configured so that liquid to be tested is contained in the open cylindrical area between the member and the hollow cylindrical container and either the hollow cylindrical container or the member is configured to be rotated.

2. The apparatus of claim 1 wherein a radius of the member is at least 90% of a radius of the open cylindrical area.

3. The apparatus of claim 1 further comprising a motor 24 to drive the rotation.

4. The apparatus of claim 1 further comprising a camera for recording the appearance of the liquid and the particulate through the transparent wall.

5. A method for testing particulate suspension in a liquid using the apparatus of claim 1, the method comprising:
introducing the liquid and a particulate to the gap;
rotating either the hollow cylindrical container or the member such that a shear field is induced between the member and the hollow cylindrical container; and
monitoring the particulate suspension of the particulate in the liquid.

6. The method of claim 5 wherein monitoring includes observing the particulate suspension of the particulate in the liquid through the transparent wall.

7. The method of claim 5 wherein observing includes computer imaging.

8. The method of claim 5 further comprising adjusting a speed of rotation.

9. The method of claim 5 wherein the liquid is a fracturing fluid and the particulate is a proppant.

10. The method of claim 5 wherein monitoring includes determining a shear rate at which insufficient shear is induced to suspend the particulate in the liquid

11. The method of claim 5 wherein monitoring includes determining the rotational velocity, and hence the shear rate, at which sufficient shear is present to suspend settled particulate.
